(19) United States
(12) Patent Application Publication
(10) Pub. No.: US 2024/0245757 A1
(43) Pub. Date: Jul. 25, 2024

(54) Use of Phospholipase A2 from Elapidae snakes in the treatment of diabetic nephropathy

(71) Applicant: Zhankai Qi, Richmond (CA)

(72) Inventor: Zhankai Qi, Richmond (CA)

(21) Appl. No.: 18/216,636

(22) Filed: Jul. 27, 2023

(30) Foreign Application Priority Data
Jun. 30, 2022 (CN) 2022107554417

(51) Int. Cl. A61K 38/46; A61P 13/02

(52) U.S. Cl. CPC A61K 38/465; A61P 13/02; C12Y 301/01004

(57) ABSTRACT

The invention relates to a pharmaceutical composition, which comprises Elapidae Phospholipase A2 and a pharmaceutically acceptable carrier thereof. This pharmaceutical composition can be used for the treatment of micro proteinuria and renal disfunction with increased ratio of urinary albumin vs creatinine of diabetes nephropathy patients, thereby controlling and delaying the pathological progress of kidney, improving the renal function, and has extremely potential for the treatment of diabetes nephropathy.

### FIELD OF THE INVENTION

The invention relates to a group of Phospholipase A2 monomer molecules which can inhibit streptozotocin (STZ)-induced diabetic nephropathy in rats and improve their renal function, belonging to the field of Biochemistry and biopharmaceutical technology.

### BACKGROUND OF THE INVENTION

Diabetes is a metabolic disorder syndrome represented by high blood glucose, its main clinical manifestation is polydipsia and polyuria with weight loss. Diabetes nephropathy is glomerulosclerosis caused by diabetes. It is a chronic complication of diabetes, which is also an important factor that causes end-stage renal disease. Delayed treatment will lead to renal failure and even death of patients. Therefore, it is necessary to improve the diagnosis of diabetes nephropathy, detect the disease as early as possible, control the progression, and ensure the safety and health of patients.

The early clinical symptoms of patients with diabetes nephropathy are not significant and can last for many years with micro proteinuria. Attention will only be raised when the disease worsens, means significant proteinuria or edema. In fact, in patients with diabetic nephropathy for years, a small amount of blood albumin will begin to leak into the urine. This is the first stage of diabetic nephropathy, called microalbuminuria. Although microalbuminuria may also be associated with hypertension, hyperlipidemia, atherosclerosis and other cardiovascular diseases, however when other biomarkers of renal function such as immunoglobulin, B2 microglobulin, α1 microglobulin and transferrin also increase significantly in urine, pathological changes at kidney level can be confirmed. [1-11]

When glomerular function decreases, β2 microglobulin will get into urine, leading to a significant increase in its content in the urine. Therefore, urine β2 microglobulin can directly reflect glomerular function. [12,13]

In the blood, after passing through the glomerular filtration membrane, α1 microglobulin is reabsorbed in the proximal tubules of the kidney, and only a small amount is eliminated from the urine. Therefore, in the urine when the content of α1 microglobulin increases, it can directly reflect the abnormal reabsorption function of renal tubules and glomerular filtration [14,15,16]. Transferrin is an iron containing protein in the plasma, which is mainly responsible for carrying iron. The negative charge of transferrin molecule is not intense, so relatively easy to pass through the charge barrier of the glomerulus, especially in the early stage of diabetes nephropathy, the negative charge of the filtration membrane is reduced, but the filter hole has not changed. The transferrin in urine can appear earlier than albumin, which can be used as a more sensitive indicator for the diagnosis of early renal disease. When the human renal function is in disorder, transferrin content will significantly increase in urine [17-21]. The G immunoglobulin is a direct indicator of the body's autoimmune response. Therefore, trace amount of β2 albumin microglobulin, α1 Microglobulin, transferrin, immunoglobulin G in human urine and other related renal function indicators can directly reflect the human renal function and disease. When there is pathological changes in kidney, urinary β2 microalbumin, α1 macroglobulin, the contents of microglobulin, transferrin and immunoglobulin G will increase significantly. Experimental test shows that simultaneous detection of albumin, β2 microglobulin, α1 microglobulin, transferrin, immunoglobulin G and other micro proteins in urine provides more reliable parameters for clinical diagnosis, as it overcomes the heterogeneity of various micro proteins. [1-3]

In further study of diabetes nephropathy, Lemann et al confirmed that the ratio of urinary protein to urinary creatinine can accurately reflect the excretion of renal protein, and can make early diagnosis of diabetes nephropathy (DN); Li Xiaoming and others also confirmed through clinical research that the ratio of protein to urinary creatinine is a sensitive indicator for early diagnosis of diabetes nephropathy (DN).

### DETAILED DESCRIPTION OF THE INVENTION

Phospholipase A2 (PLA2) is a widely distributed enzyme family which exists in various animal tissues, especially in the snake venom and the pancreatic secretion of mammals. They all have a common basic function, that is, they use biomembrane phospholipids as natural substrates to catalyze the hydrolysis of the acyl bond on the sn-2 position of Glycerophospholipid, produce lysophospholipids and fatty acids, and participate in the metabolism of phospholipids. Snake venom Phospholipase A2 is very similar to mammalian secretory PLA2 in structure and hydrolysis function.

Our animal model study found for the first time that Elapidae snake Phospholipase A2 can inhibit the micro proteinuria of diabetes nephropathy induced by Streptozotocin (STZ), reverse the increase of the ratio of urinary microalbumin to urinary creatinine (UACR), and improve the renal function of diabetic nephropathy. Elapidae Phospholipase A2, including that of Naja atra, Bungarus multicinctus, Naja naja, Naja kaouthia and Bungarus fasciatus, their Phospholipases A2 have almost reached the same experimental results in the treatment of micro proteinuria and renal function damage in rats with diabetes nephropathy. In terms of protein structure, they all belong to type IA PLA2 structure, which is mainly from that of Naja, Bungarus multicinctus, and Bungarus fasciatus, with a molecular weight of 13^{˜}14 KD and the molecule contains 7 disulfide bonds, and all of which are typical disulfide bonds at positions 11 and 77 as Type IA PLA2. Their mature proteins have high homology in amino acid sequences, with 118 or 119 amino acid residues and their common functional structure enables them to have common biological activity. [24-27]. The amino acid sequences (FASTA) of their mature proteins are as follows:

Naja atra PLA2
(SEQ ID No. 1)
nly qfknmiqctv psrswwdfad ygcycgrggs
gtpvddldrc cqvhdhcyne aekisgcwpy
sktysyecsq gtltckggnn acaaavcdcd
rlaaicfaga pynnnnynid Ikarcq
Naja atra PLA2
(SEQ ID No. 2)
nlyqfknmiq ctvpsrswwd fadygcycgr
ggsgtpvddl drccqvhdnc yneaekisgc
wpyfktysye csqgtltckg gnnacaaavc
dcdrlaaicf agapyndndy ninlkarcq
Naja atra PLA2
(SEQ ID No. 3)
nly qfknmiqctv psrswwdfad ygcycgrggs
gtpvddldrc cqvhdncyne aekisgcwpy
fktysyecsq gtltckggnn acaaavcdcd
rlaaicfaga pynnnnynid Ikarcq
Naja kaouthia PLA2
(SEQ ID No. 4)
nly qfknmiqctv psrswwdfad ygcycgrggs
gtpvddldrc cqvhdncyne aekisgcwpy
fktysyecsq gtltckggnn acaaavcdcd
rlaaicfaga pynnnnynid Ikarcq
Naja kaouthia PLA2
(SEQ ID No. 5)
nly qfknmiqctv pnrswwdfad ygcycgrggs
gtpvddldrc cqvhdncyne aekisrcwpy
fktysyecsq gtltckgdnd acaaavcdcd
rlaaicfaga pynnnnynid Ikarcq
Naja kaouthia PLA2
(SEQ ID No. 6)
nly qfknmiqctv pnrswwdfad ygcycgrggs
gtpvddldrc cqvhdhcyne aekisrcwpy
fktysyecsq gtltckgdnd acaaavcdcd
rlaaicfaga pynrndynid Ikarcq
Naja kaouthia PLA2
(SEQ ID No. 7)
nly qfknmiqctv psrswwdfad ygcycgrggs
gtpvddldrc cqvhdncyne aekirgcwpy
mktysyecsq gtltckggnn acaaavcdcd
rlaaicfaga pyndnhynid Ikarcq`
Naja naja PLA2
(SEQ ID No. 8)
nly qfknmvqctv pnrswwdfad ygcycgrggs
gtpvddldrc cqvhdncyge aekisrcwpy
fktysyecsq gtltckggnn acaaavcdcd
rlaaicfaga pyndnnynid Ikarcq
Naja naja PLA2
(SEQ ID No. 9)
nly qfknmvqctv pnrswwhfad ygcycgrggs
gtpvddldrc cqihdncyne aekisrcwpy
fktysyecsq gtltckggnn acaaavcdcd
rlaaicfaga pyndnnynid Ikarcq
Naja naja PLA2
(SEQ ID No. 10)
nly qfknmvqctv pnrswwhfad ygcycgrggs
gtpvddldrc cqihdhcyne aekisrcwpy
sktysyecsq gtltckggnn acaaavcdcd
rlaaicfaga pyndndynid Ikarcq
Bungarus multicinctus PLA2
(SEQ ID No. 11)
nly qfknmivcag trpwigyvny gcycgaggsg
tpvdeldrcc yvhdncygea ekipgcnpkt
ktysytctkp nltctdaagt carivcdcdr
taaicfaaap yninnfmiss sthcq
Bungarus fasciatus PLA2
(SEQ ID No. 12)
nly qfknmiecag trtwlayvky gcycgpggtg
tpldeldrcc qthdhcydna kkfgncipyl
ktyvytcnkp ditctgakgs cgrtvcdcdr
aaaicfaaap ynlanfgidk ekhcq

In some embodiments, the Elapidae phospholipase A2 of SEQ ID No.1 shows the ability of decreasing the micro-proteinuria and the ratio of urinary microalbumin to urinary creatinine (UACR) of diabetes nephropathy.

In some embodiments, the Elapidae phospholipase A2 of SEQ ID No.2 shows the ability of decreasing the micro-proteinuria and the ratio of urinary microalbumin to urinary creatinine (UACR) of diabetes nephropathy.

In some embodiments, the Elapidae phospholipase A2 of SEQ ID No.3 shows the ability of decreasing the micro-proteinuria and the ratio of urinary microalbumin to urinary creatinine (UACR) of diabetes nephropathy.

In some embodiments, the Elapidae phospholipase A2 of SEQ ID No.4 shows the ability of decreasing the micro-proteinuria and the ratio of urinary microalbumin to urinary creatinine (UACR) of diabetes nephropathy.

In some embodiments, the Elapidae phospholipase A2 of SEQ ID No.5 shows the ability of decreasing the micro-proteinuria and the ratio of urinary microalbumin to urinary creatinine (UACR) of diabetes nephropathy.

In some embodiments, the Elapidae phospholipase A2 of SEQ ID No.6 shows the ability of decreasing the micro-proteinuria and the ratio of urinary microalbumin to urinary creatinine (UACR) of diabetes nephropathy.

In some embodiments, the Elapidae phospholipase A2 of SEQ ID No.7 shows the ability of decreasing the micro-proteinuria and the ratio of urinary microalbumin to urinary creatinine (UACR) of diabetes nephropathy.

In some embodiments, the Elapidae phospholipase A2 of SEQ ID No.8 shows the ability of decreasing the micro-proteinuria and the ratio of urinary microalbumin to urinary creatinine (UACR) of diabetes nephropathy.

In some embodiments, the Elapidae phospholipase A2 of SEQ ID No.9 shows the ability of decreasing the micro-proteinuria and the ratio of urinary microalbumin to urinary creatinine (UACR) of diabetes nephropathy.

In some embodiments, the Elapidae phospholipase A2 of SEQ ID No.10 shows the ability of decreasing the micro-proteinuria and the ratio of urinary microalbumin to urinary creatinine (UACR) of diabetes nephropathy.

In some embodiments, the Elapidae phospholipase A2 of SEQ ID No.11 shows the ability of decreasing the micro-proteinuria and the ratio of urinary microalbumin to urinary creatinine (UACR) of diabetes nephropathy.

In some embodiments, the Elapidae phospholipase A2 of SEQ ID No.12 shows the ability of decreasing the micro-proteinuria and the ratio of urinary microalbumin to urinary creatinine (UACR) of diabetes nephropathy.

In the current invention, said Phospholipases A2 from cobra snake of Naja atra, Naja kaouthia and Naja naja of SEQ ID No1-No10, their aminoacid sequences of mature proteins have at least 96% of the identity.

In an embodiment, the Phospholipase A2 is isolated from nature snake venom.

In an embodiment, the Phospholipase A2 is synthesized from chemical polypeptides.

In an embodiment, the Phospholipase A2 is produced by recombinant technology.

In an embodiment, the Phospholipase A2 is administered once a day for 15-120 consecutive days.

In an embodiment, the Phospholipase A2 is administered once every 12 hours, twice a day, and continuously for 15-120 days.

In an embodiment, the Phospholipase A2 is administered every 4 hours, 3 times a day, and continuously for 15-120 days.

In some embodiments, the dosage of the Phospholipase A2 is from 1-350 μg/kg once.

In an embodiment, the dosage of the Phospholipase A2 is 80 μg/kg once.

In an embodiment, the dosage of the Phospholipase A2 is 40 μg/kg once.

In an embodiment, the dosage of the Phospholipase A2 is 20 μg/kg once.

In an embodiment, the dosage form of the Phospholipase A2 is a sublingual membrane form.

In an embodiment, the dosage form of the Phospholipase A2 is an oral dosage form.

In an embodiment, the dosage form of the Phospholipase A2 is an injection form.

In an embodiment, the dosage form of the Phospholipase A2 is a nasal spray type.

In some embodiments, the pharmaceutically acceptable carrier are selected from. group consisting of protein stabilizers, membrane excipients, mucosal penetration enhancer, co solvents, or solvents.

In an embodiment, the protein stabilizer is mannitol.

In some embodiments, the membrane excipients are propylene glycol, polyethylene glycol, and hydroxypropyl β Cyclodextrin, Polysorbate, hydroxypropyl methylcellulose (HPMC), methylcellulose, xanthan gum.

In some embodiments, the mucosal penetration enhancers are laurazone, poloxamer, camphor, and dexcamphor.

In an embodiment, the co solvent is propylene glycol.

Another advantage of the invention is for product manufacturing, because the phospholipase A2 monomer molecule from Elapidae disclosed by the invention has a clear amino acid sequence, it can be produced through genetic engineering, which solves the practical problem of scarcity of snake venom resources; even if we continue to obtain PLA2 through the separation and purification of natural snake venom, it is easier to control the quality and purity due to the identified amino acid sequence in the process, setting up a necessary basis for the drug development of monomer from snake venom.

The above scheme is further described below in combination with specific embodiments. It should be understood that these embodiments are used to illustrate the invention and not to limit the scope of the invention.

### EXAMPLES

### Example A: Obtaining PLA2 of SEQ ID No1 from Naja atra

1. Separation and Purification of Phospholipase A2 from Naja atra

Dissolve 1 g of Naja atra crude venom in 25 ml 0.025 mol ph6 0 ammonium acetate buffer, centrifugation at low temperature, and take the supernatant; Use 0.025 mole ph6 0 ammonium acetate solution to balance TSK cm-650 (m) column; After loading the sample, 0.1^{˜}0.5 mol and 0.7^{˜}1.0 mol, pH5. 9 ammonium acetate buffer was used for elution in two compartment gradient, and the UV detection parameter was set at 280 nm; Elution flow rate: 48 ml/h; Various toxin components were collected according to the recorded spectrum, and 12 protein peaks were washed out from the collected solution.

### 2. Sequencing

The protein with a molecular weight of 13-14 KD and the first 8 amino acids residue of nlyqfknm at the N-terminus was sequenced, finally the amino acid sequence of Phospholipase A2 of Naja atra was obtained.

The amino acid sequence of the primary structure of Phospholipase A2 of Naja atra (SEQ ID No. 1) in the form of Fasta is: nly qfknmiqctv psrswwdfad ygcycgrggs gtpvddldrc cqvhdhcyne aekisgcwpy sktysyecsq gtltckggnn acaaavcdcd rlaaicfaga pynnnnynid Ikarcq.

Phospholipase A2 of SEQ ID No.2-SEQ ID No.12 can be obtained by the same method.

Example B: The effect of Phospholipase A2 from Naja atra (SEQ ID No. 1) on micro proteinuria and urinary microalbuminuria creatinine ratio in diabetes nephropathy rats induced by Streptozotocin (STZ).

Streptozotocin (STZ) induced diabetes nephropathy in rats can cause typical diabetes nephropathy, and the pathological changes of its animal model are similar to human diabetes nephropathy micro pathological changes.

### 1. Experimental Animals, Modeling and Grouping

30 rat for experimental testing, 10 in the treatment group; 10 in the model group; and
in control group of normal rats, details are as follows:
40 male SD rats weighing 160-180 g were randomly divided into a control group of 10 and a model group of 30. Twenty rats survived after successful modeling were randomly selected and divided into Naja atra Phospholipase A2 treatment group (10 rats) and model group (10 rats), the rest were out of the experiment. The specific modeling method is that the rats in the model group eat and drink normally, each rat is injected with 0.5 ml Freund's Complete Adjuvant (CFA) intraperitoneally first, Streptozotocin (STZ) solution is injected intraperitoneally the next day, and 0.1 mmol/L of PH4.5 citric acid buffer is mixed with Streptozotocin (STZ) into a concentration of 1% solution before experiment, and is injected intraperitoneally with dose of 55 mg/kg. One week later, the tail vein blood of the modeling group is taken to detect blood glucose, and if the random blood glucose is maintained at above 16.7 mmol/L, and urine glucose 3+^{˜}4+, it is considered as a successful diabetes model. Phospholipase A2 treatment group was given Naja atra Phospholipase A2 20 μg/kg by gavage once a day for 8 weeks. The model group and control group were gavaged with physiological saline once a day for 8 consecutive weeks.

### 2. Biomarkers and Testing Methods

After the last treatment, the rats were placed in a metabolic cage to collect 10 ml of urine, centrifuged at 3500 r/min for 10 minutes, and the supernatant was extracted into a frozen storage tube. The supernatant was stored in a refrigerator at −80° C. for future use. Urine α1 microglobulin was detected using enzyme-linked immunosorbent assay, β2 microglobulin, microalbumin, transferrin, immunoglobulin G (IgG) and creatinine (Cr) were tested according to the instructions of the ELISA kit.

### 3. Results

Table 1 shows the experimental results of the influence of Naja atra Phospholipase A2 (PLA2) of ID No. 1 on renal function indexes of rats with Streptozotocin (STZ) induced diabetic nephropathy.

TABLE 1
(x ± SD, n = 10)

| Groups | UALB (μg/ml) | α 1-MG (μg/ml) | β2-MG (ng/ml) | TRF (μg/ml) | IgG (μg/ml) | CR (μmol/1) | UACR (mg/μmol) |
|---|---|---|---|---|---|---|---|
| Control | 0.36 ± 0.10 | 0.38 ± 0.09 | 6.57 ± 1.36 | 0.04 ± 0.006 | 0.12 ± 0.04 | 196.00 ± 20.33 | 1.93 |
| Model | 1.01 ± 0.14 ## | 1.12 ± 0.23 ## | 32.29 ± =4.95 # | 0.20 ± 0.07 # | 0.56 ± 0.19 ## | 128.00 ± 14.87 # | 8.76 |
| PLA 2 | 0.59 ± 0.15 | 0.79 ± 0.16 | 27.30 ± =4.99 | 0.13 ± 0.039 | 0.30 ± 0.13 | 179.00 ± 16.88 | 4.43 |

i. The effect of Phospholipase A2 on urinary microalbumin (UALB) of rats with diabetes nephropathy induced by Streptozotocin (STZ).

Compared with the control group, the urinary microalbumin (UALB) of rats in the two groups after modeling were significantly increased; Compared with the model group, the urinary microalbumin of Phospholipase A2 treatment group was significantly reduced. ## means P<0.01 when the Phospholipase A2 treatment group is compared with the model group.
ii. The effect of Phospholipase A2 on the urine α1 Microglobulin (α1-MG) of rats with diabetes nephropathy induced by Streptozotocin (STZ), Compared with the control group, the urine α1 Microglobulin of the two groups of rats after modeling were significantly increased; Compared with the model group, the urine α1 Microglobulin of Phospholipase A2 treatment group was a significant decreased. ## means P<0.01 when the Phospholipase A2 treatment group is compared with the model group.
iii. The effect of Phospholipase A2 on the urine β2-microglobulin (β2-MG) of rats with diabetes nephropathy induced by Streptozotocin (STZ). Compared with the control group, the β2-microglobulin (β2-MG) of rats in the two modeling groups were significantly increased; Compared with the model group, the β2-microglobulin (β2-MG) of Naja Phospholipase A2 treatment group was significantly reduced. # means P<0.05 when the Naja Phospholipase A2 treatment group is compared with the model group.
iv. The effect of Phospholipase A2 on urinary transferrin (TRF) of rats with diabetes nephropathy induced by Streptozotocin (STZ). Compared with the control group, the urinary transferrin (TRF) of rats in the two modeling groups were significantly increased; Compared with the model group, the urinary transferrin (TRF) of Naja Phospholipase A2 treatment group was significantly reduced. # means P<0.05 when the Phospholipase A2 treatment group is compared with the model group.
v. The effect of Phospholipase A2 on urinary immunoglobulin (IgG) of rats with diabetes nephropathy induced by Streptozotocin (STZ). Compared with the control group, the urinary immunoglobulin (IgG) of rats in the two groups after modeling were significantly increased; Compared with the model group, the urinary immunoglobulin (IgG) of Phospholipase A2 treatment group was significantly reduced. ## means P<0.01 when the Phospholipase A2 treatment group is compared with the model group.
vi. The effect of Phospholipase A2 on urinary creatinine (Cr) of rats with diabetes nephropathy induced by Streptozotocin (STZ). Compared with the control group, the urinary creatinine (Cr) of rats in the two groups after modeling were significantly increased; Compared with the model group, the urinary creatinine (Cr) of Phospholipase A2 treatment group was significantly reduced. # means P<0.05 when the Phospholipase A2 treatment group is compared with the model group.
vii. The effect of Phospholipase A2 on ratio of urinary microalbumin to urinary creatinine (UACR) of rats with diabetes nephropathy induced by Streptozotocin (STZ). Compared with the control group, the ratio of urinary microalbumin to urinary creatinine (UACR) of rats in the two groups after modeling were significantly increased; Compared with the model group, the ratio of urinary microalbumin to urinary creatinine (UACR) of Phospholipase A2 treatment group was significantly reduced. P<0.05.

### REFERENCES

1. Zhang Guiyun. The Application of Urinary Microprotein Determination in Early Diagnosis of Nephropathy. Contemporary Medicine 2013000 (027)-146-146
2. Gao Xuehui. Clinical Application of Urinary Microprotein in Early Renal Injury. Shaanxi J Lab Sci Volume 16 Issue 2, May 2001
3. Song Xue et al., The clinical observation and diagnostic value of different urine protein tests in kidney diseases. International Journal of Urinary system 2012, Volume 32, Issue 2, March
4. Zhu Xiaobin. The value of urine and renal function test in the clinical diagnosis of early diabetes nephropathy. Chinese Medical Guidelines 2017015 (002)—
5. Wang Xiao. The clinical significance of urinary microprotein in judging renal damage in diabetes. Journal of Zhejiang Chinese Medical University, 2008, 32 (6): 751-752
6. Su Cai et al., The value of blood and urine microprotein determination in early diagnosis of diabetes nephropathy. Journal of Radioimmunology, 2001, 14 (4): 502
7. Duan Li et al., Analysis of urinary trace protein types and levels in patients with primary nephrotic syndrome and lupus nephritis. Chinese Tropical Medicine 2003003 (006)-771-772
8. Yang Fang et al., The Relationship between Urinary Microprotein Changes and Renal Damage in Reflux Nephropathy. Journal of Jinan University natural science and medicine. 2000021 (004)-104-106
9. Shen Shunbiao et al., Clinical significance of urinary microprotein content in IgA nephropathy. Chinese Journal of Nephrology 1995000 (001) −34^{˜}34
10. Teng Shoufeng et al., The value of urinary microprotein in the early diagnosis of renal injury in diabetes. Chinese Journal of Misdiagnostics 2009009 (034)
11. Shao Aihua et al., Observation of urine trace protein determination results in various kidney disease patients. Journal of Zhejiang University of Traditional Chinese Medicine 2003027 (005)-31^{˜}32
12. Huo Meifeng. The value of β_2-microglobulin in the early diagnosis of diabetes nephropathy Chinese General Medicine. 2012 (12): 119-120
13. Kang Ping et al., The significance of β2 microglobulin in early renal damage of diabetes. Heilongjiang Medicine 2008
14. Lu Qingyun et al., The clinical significance of α_1, β2-microglobulin in early renal damage in primary hypertension. [J] Western Traditional Chinese Medicine Medicine, 2014:131-133
15. Wang Chunwei. The clinical value of urine α1 microglobulin in diabetes nephropathy. Journal of Practical Medical Technology 2007014 (025)
16. Yan Jingchun. The clinical significance of urine α1 microglobulin detection in secondary kidney disease. Medical Journal of Coal in China, 2014017 (006)
17. Wei Wenfeng. The significance of urinary trace transferrin in the early diagnosis of diabetes nephropathy. Labeled Immunoassay and clinical, 2002
18. Li Xiaoxia et al., Comparison of urinary trace transferrin and albumin in early glomerular injury. Journal of Medical Forum, 2010
19. Zhu Guangbo et al., Clinical significance of urinary trace transferrin in patients with diabetes. Shaanxi Medical Journal, 1999
20. Dong Hui et al., Clinical significance of combined detection of microglobulin and transferrin in early diagnosis of diabetes nephropathy. Tianjin Medical University Journal, 2002, 8 (2): 253
21. Martin et al., Early prediction of urinary transport and glomerular dysfunction. Annual Journal of Clinical Biochemistry, 1988, 5 (Supplement): 158
22. Lehman et al., Study the role of protein in health and disease by measuring the ratio of urinary protein to creatinine. Clinical chemistry, 1987, 33:297]
23. Li Xiaoming et al., The diagnostic value of urinary microalbumin/creatinine ratio in diabetes nephropathy. Hubei Journal of Preventive healthcare Volume 12, Issue 3, 2001
24. Berg et al., Interface enzymology: secretory Phospholipase A2 paradigm. Chemical Review 2001 101 (9): 2613-54
25. Yang Yaping et al., Research progress of cobra venom secretory Phospholipase A2. Chinese Pharmacological Bulletin 2005, 21 (9):
26. Shibbir Ahmed Khan and Marc A. Ilies., The Phospholipase A2 Superfamily: Structure, Isozymes, Catalysis, Physiologic and Pathologic Roles. Int J Mol Sci. 2023 January; 24(2): 1353.
27. Six D. A., Dennis E. A. The Expanding Superfamily of Phospholipase A2 Enzymes: Classification and Characterization. Biochim. Et Biophys. Acta BBA-Mol. Cell Biol. Lipids. 2000; 1488:1-19. doi: 10.1016/S1388-1981(00)00105-0

1. A method for treating diabetic nephropathy in a mammal. Said method comprising administering to a mammal in need thereof a pharmaceutical composition of a therapeutically effective amount of elapidae Phospholipase A2, and a pharmaceutically acceptable carrier for use in treatment of diabetic nephropathy.

2. According to claim 1, wherein the diabetic nephropathy means proteinuria, impairment of renal function, and increased ratio of urine albumin to urine creatinine which is beyond the normal ratio range defined by medicine standard.

3. According to claim 2, wherein the diabetic nephropathy proteinuria is further characterized in that it consists of increase of one, or pleural, or all of the following biomarkers including albumin, immunoglobulins, β2 microglobulin, α1 microglobulin, and transferrin, and wherein the increased level of said biomarker is over the normal range of medical diagnosis.

4. (canceled)

5. (canceled)

6. The elapidae Phospholipase A2 of claim 1, wherein it is an elapidae Phospholipase A2 having the amino acid sequence selected from the group consisting of SEQ ID No.1 to SEQ ID No.12; or elapidae Phospholipase A2 homologues having at least 96% or more identity with the elapidae Phospholipase A2 of SEQ ID No. 1 to SEQ ID No.10, and the biological function of the elapidae Phospholipase A2 homologues is the same as, or similar to that of the elapidae Phospholipase A2 of the amino acid sequence ID No. 1 to SEQ ID No. 10.

7. The elapidae Phospholipase A2 according to claim 6, is further characterized in that they are derived from natural snake venoms, or synthesized from chemical polypeptides, or obtained from prokaryotic or eukaryotic hosts using recombinant technology.

8. (canceled)

9. The elapidae Phospholipase A2 according to claim 1, is further characterized in that it includes the elapidae Phospholipase A2 combined with a compound that extends the half-life of a polypeptide, such as polyethylene glycol, or the elapidae Phospholipase A2 polypeptide formed by fusion of a fatty chain, or by fusing an additional amino acid sequence to its polypeptide sequence. As described herein, these derivatives, or analogs are within the scope of those skilled in the art.

10. According to claim 1, wherein, the method of administration comprises intravenous, intramuscular, subcutaneous, intra-articular, oral, sublingual, and nasal administration.

11. The dose of Phospholipase A2 of claim 1 includes from 1 μg/Kg to 350 μg/kg each time, and the administration frequency ranges from once a day to multiple times a day, or multiple times a year.


SEQUENCE LISTING (12 sequences)

SEQ ID NO: 1 - protein, 119 aa, NAJA ATRA
NLYQFKNMIQCTVPSRSWWDFADYGCYCGRGGSGTPVDDLDRCCQVHDHCYNEAEKISGCWPYSKTYSYECSQGTLTCKGGNNACAAAVCDCDRLAAICFAGAPYNNNNYNIDLKARCQ

SEQ ID NO: 2 - protein, 119 aa, NAJA ATRA
NLYQFKNMIQCTVPSRSWWDFADYGCYCGRGGSGTPVDDLDRCCQVHDNCYNEAEKISGCWPYFKTYSYECSQGTLTCKGGNNACAAAVCDCDRLAAICFAGAPYNDNDYNINLKARCQ

SEQ ID NO: 3 - protein, 119 aa, NAJA ATRA
NLYQFKNMIQCTVPSRSWWDFADYGCYCGRGGSGTPVDDLDRCCQVHDNCYNEAEKISGCWPYFKTYSYECSQGTLTCKGGNNACAAAVCDCDRLAAICFAGAPYNNNNYNIDLKARCQ

SEQ ID NO: 4 - protein, 119 aa, NAJA KAOUTHIA; identical to SEQ ID NO: 3

SEQ ID NO: 5 - protein, 119 aa, NAJA KAOUTHIA
NLYQFKNMIQCTVPNRSWWDFADYGCYCGRGGSGTPVDDLDRCCQVHDNCYNEAEKISRCWPYFKTYSYECSQGTLTCKGDNDACAAAVCDCDRLAAICFAGAPYNNNNYNIDLKARCQ

SEQ ID NO: 6 - protein, 119 aa, NAJA KAOUTHIA
NLYQFKNMIQCTVPNRSWWDFADYGCYCGRGGSGTPVDDLDRCCQVHDHCYNEAEKISRCWPYFKTYSYECSQGTLTCKGDNDACAAAVCDCDRLAAICFAGAPYNRNDYNIDLKARCQ

SEQ ID NO: 7 - protein, 119 aa, NAJA KAOUTHIA
NLYQFKNMIQCTVPSRSWWDFADYGCYCGRGGSGTPVDDLDRCCQVHDNCYNEAEKIRGCWPYMKTYSYECSQGTLTCKGGNNACAAAVCDCDRLAAICFAGAPYNDNHYNIDLKARCQ

SEQ ID NO: 8 - protein, 119 aa, NAJA NAJA
NLYQFKNMVQCTVPNRSWWDFADYGCYCGRGGSGTPVDDLDRCCQVHDNCYGEAEKISRCWPYFKTYSYECSQGTLTCKGGNNACAAAVCDCDRLAAICFAGAPYNDNNYNIDLKARCQ

SEQ ID NO: 9 - protein, 119 aa, NAJA NAJA
NLYQFKNMVQCTVPNRSWWHFADYGCYCGRGGSGTPVDDLDRCCQIHDNCYNEAEKISRCWPYFKTYSYECSQGTLTCKGGNNACAAAVCDCDRLAAICFAGAPYNDNNYNIDLKARCQ

SEQ ID NO: 10 - protein, 119 aa, NAJA NAJA
NLYQFKNMVQCTVPNRSWWHFADYGCYCGRGGSGTPVDDLDRCCQIHDHCYNEAEKISRCWPYSKTYSYECSQGTLTCKGGNNACAAAVCDCDRLAAICFAGAPYNDNDYNIDLKARCQ

SEQ ID NO: 11 - protein, 118 aa, Bungarus multicinctus
NLYQFKNMIVCAGTRPWIGYVNYGCYCGAGGSGTPVDELDRCCYVHDNCYGEAEKIPGCNPKTKTYSYTCTKPNLTCTDAAGTCARIVCDCDRTAAICFAAAPYNINNFMISSSTHCQ

SEQ ID NO: 12 - protein, 118 aa, Bungarus fasciatus
NLYQFKNMIECAGTRTWLAYVKYGCYCGPGGTGTPLDELDRCCQTHDHCYDNAKKFGNCIPYLKTYVYTCNKPDITCTGAKGSCGRTVCDCDRAAAICFAAAPYNLANFGIDKEKHCQ